(19) United States
(12) Patent Application Publication
(10) Pub. No.: US 2004/0243203 A1
(43) Pub. Date: Dec. 2, 2004

(54) Temperature retaining pad with removable cover

(76) Inventor: Kim D. Lavine, Grand Haven, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/887,730

(22) Filed: Jul. 9, 2004

Related U.S. Application Data
(63) Continuation-in-part of application No. 10/064,840, filed on Aug. 22, 2002, now abandoned.
(60) Provisional application No. 60/354,399, filed on Feb. 4, 2002.

(51) Int. Cl. A61F 7/00

(52) U.S. Cl. 607/114

(57) ABSTRACT

A therapeutic device comprising an enclosed member and a cover. The enclosed member has a plurality of kernels of seed corn therein. The cover encloses the enclosed member. The cover has a slit for the insertion and removal of the enclosed member. The kernels of seed corn are capable of being exothermic when heated or endothermic when cooled. The enclosed member can be removed from the cover and the cover can be washed to clean the cover.

### CROSS-REFERENCE TO RELATED APPLICATIONS

This application is a continuation-in-part of U.S. patent application Ser. No. 10/064,840, filed on Aug. 22, 2002, which claims priority under 35 U.S.C. 119(e) to U.S. Provisional Patent Application No. 60/354,399, filed on Feb. 4, 2002, the entire disclosures of which are incorporated herein by reference.

### BACKGROUND OF THE INVENTION

The field of endeavor to which this invention pertains is the therapeutic health and personal care field, and more particularly, the present invention relates to heating and cooling pads.

Existing heating pads include electric heating pads and heatable silicone packs. Such heating pads are typically not very attractive and may require continued supply of electricity to produce heat.

The inventor's own U.S. patent application Ser. No. 10/064,840 discloses a heating and cooling pad that may utilizes various endothermic/exothermic organic materials enclosed in a bag. This patent application further discloses that it is preferred to use either feed corn or seed corn as the organic material. The benefits of using an organic material are that they provide natural, reusable alternatives to heating pads and heatable silicone packs. The invention described in this patent application met the need to provide consumers with a natural, low-cost alternative to heating pads and expensive heatable silicone packs. Additionally, the invention described in this patent application provides consumers with an attractive alternative to heating pads and heatable silicone packs.

The inventor initially utilized feed corn as the endothermic/exothermic organic material due to its low cost. Through subsequent research, however, the inventor discovered that feed corn produces a pungent odor when heated and did not retain and release moisture consistently through extended use. Further, feed corn is more typically stored exposed in warehouses, where it is prone to disease, mixing with dust and foreign matter, and to damage by rodents, insects, and mold.

Accordingly, a temperature retaining bag solving the aforementioned disadvantages and having the aforementioned advantages is desired.

### SUMMARY OF THE INVENTION

One aspect of the present invention is to provide a therapeutic device comprising an enclosed member and a cover. The enclosed member has a plurality of kernels of seed corn therein. The cover encloses the enclosed member. The cover has a slit for the insertion and removal of the enclosed member. The kernels of seed corn are capable of being exothermic when heated or endothermic when cooled.

Another aspect of the present invention is to provide a method of administering heat comprising the steps of providing an enclosed member having kernels of seed corn therein, and heating the enclosed member to heat the kernels of seed corn therein. The method also includes the steps of placing the enclosed member within a cover by inserting the cover into a slit in the cover and applying the cover with the enclosed member therein to an area to be heated.

Yet another aspect of the present invention is to provide a method of administering cold comprising the steps of providing an enclosed member having kernels of seed corn therein, and cooling the enclosed member to cool the kernels of seed corn therein. The method also includes the steps of placing the enclosed member within a cover by inserting the cover into a slit in the cover and applying the cover with the enclosed member therein to an area to be cooled.

The object of the invention is to provide a new, unique and effective way to relieve pain and muscle soreness, to relieve tension, and to provide an overall sense of enjoyment and comfort, while also answering a need of consumers for an aesthetically attractive product.

The inventor has discovered that seed corn, because of the density and weight of the kernel, was an excellent conductor of both heat and moisture, and that the seed corn kernel has a unique ability to retain and transmit both heat and moisture for long periods of time, up to two hours or more depending on environmental conditions, (e.g., under the blankets of a bed, or between a user's back and a leather chair). The corn bag can be reheated indefinitely without an appreciable loss of heat or moisture quality, and on average, costs significantly less.

The uniqueness and novelty of the present invention is significantly distinguished by the decorative, designer-quality fabrics that encase the corn bags. This added distinction of decorative, designer-quality fabric coverings further distinguishes the present invention significantly from aesthetically unattractive heating pads and silicone packs.

The seed corn filled fabric pillow provides consumers with a unique and novel invention. It is unique and novel in its practical process application: the placement of seed corn in a bag for use as a transmitter of moist heat, for therapeutic relief of pain, muscle strain, tension relief, and for an overall sense of enjoyment and comfort. It is also unique and novel in its aesthetic application: a decorative covering of designer-quality cotton/flannel fabrics distinguishes it from aesthetically unattractive heating pads and silicone packs.

Advantages of the invention include a penetrating moist heat not found in heating pads or any silicone packs intended for application to the body for therapeutic use. The density of the corn kernel has a unique ability to retain and transmit both heat and moisture for an extended period of time; this advantage distinguishes corn pillows from heating pads and silicone packs intended for application to the body for therapeutic use.

The corn bag may be heated and taken to bed without the fear of electrical fires associated with heating pads, or possible burns, also associated with bed use of heating pads. The corn bag provides extended heat and therapeutic relief for up to two hours or more depending on environmental conditions. The corn bag provides an economical alternative to expensive silicone packs and heating pads. The corn bag, when encased in designer quality fabrics, provides consumers with an attractive alternative to aesthetically unappealing silicone packs and heating pads. The corn bag may be reused indefinitely without an appreciable loss of heat or moisture quality. The cost of use of the corn bag is significantly lower than the electrical costs associated with use of a heating pad.

These and other features, advantages, and objects of the present invention will be further understood and appreciated by those skilled in the art by reference to the following specification, claims and appended drawings.

### BRIEF DESCRIPTION OF THE DRAWINGS

FIG. 1 is a perspective exploded view of a therapeutic device of the present invention.

FIG. 2 is a perspective view of the therapeutic device of the present invention.

FIG. 3 is a perspective view of a second embodiment of the therapeutic device of the present invention.

FIG. 4 is a cross-section view of the second embodiment of the therapeutic device of the present invention.

FIG. 5 is a perspective view of a third embodiment of the therapeutic device of the present invention.

FIG. 6 is a flow diagram showing the method of using the therapeutic device to apply heat.

FIG. 7 is a flow diagram showing the method of using the therapeutic device to apply cold.

### DETAILED DESCRIPTION OF THE PREFERRED EMBODIMENTS

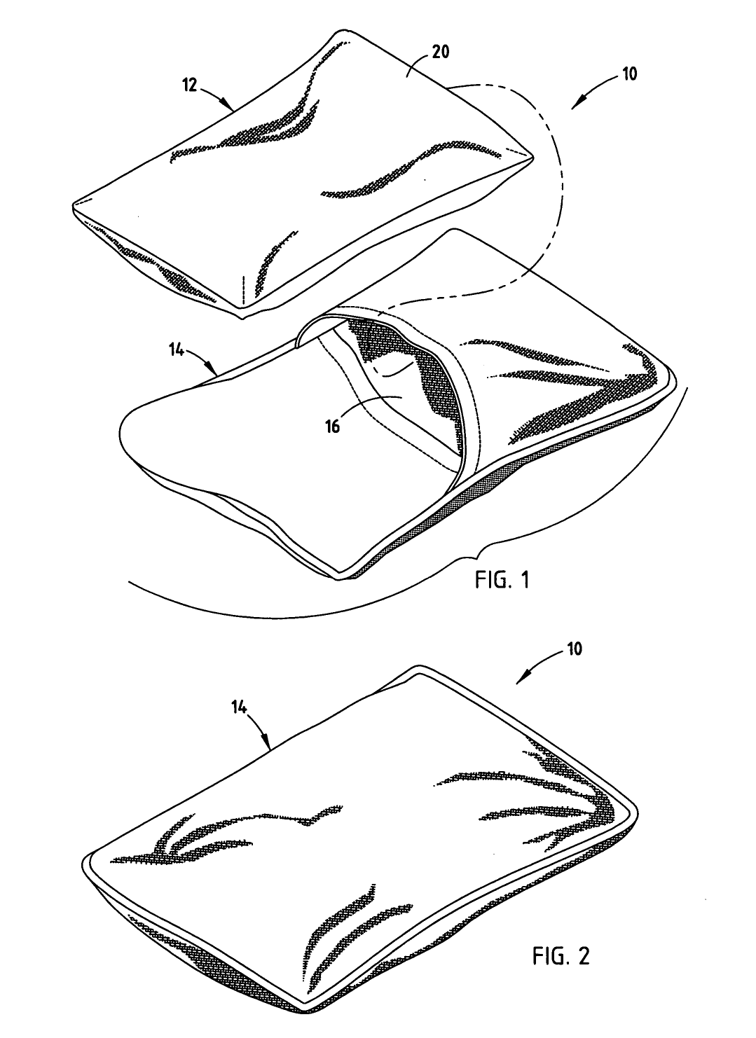

For purposes of description herein, the terms “upper,” “lower,” “right,” “left,” “rear,” “front,” “vertical,” “horizontal,” and derivatives thereof shall relate to the invention as orientated in FIG. 1. However, it is to be understood that the invention may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The reference number 10 (FIGS. 1 and 2) generally designates a therapeutic device comprising an enclosed member or bag 12 and a cover or pillowcase 14. The enclosed member 12 has a plurality of kernels of seed corn therein. The cover 14 encloses the enclosed member 12. The cover 14 has a slit 16 for the insertion and removal of the enclosed member 12. The kernels of seed corn are capable of being exothermic when heated or endothermic when cooled. The enclosed member 12 can be removed from the cover 14 and the cover 14 can be washed to clean the cover 14.

Preferably, the bag 12 is made of cloth 20. The cloth bag is constructed with a sewing machine or serger, and once filled with corn, is sewn shut with a sewing machine or serger.

The exact dimension of the therapeutic device 10, as well as the amount and weight of feed and/or seed corn, varies, as different sized pillows 14 are made for different targeted areas on the body.

Seed corn kernels offer surprising and unexpected results over feed corn. Feed corn is corn that has been dried for use as animal feed, e.g., cattle. Feed corn has been used primarily as an agricultural feed. As stated above, feed corn often includes other parts of the corn stalk and the feed corn kernels are mostly broken open, which causes the feed corn to release a more pungent odor when heated and to not retain moisture after several uses. Seed corn, on the other hand, consists of corn kernels that have been dried, sorted and cleaned for the purpose of planting. The differences between the processing methods used to produce seed corn and feed corn are discussed further below. As a result of the extra processing steps, seed corn is much more expensive than feed corn (on the order of a twenty-fold increase in cost).

Seed corn fields are typically irrigated, fertilized, and sprayed for disease and insects more extensively than feed corn fields. Seed corn is harvested on the ear with sweet corn husking beds that cause less damage to the kernels. The seed is then dried on the ear at low heat with a forced air system to ensure viable seed, strong seed coats, and less damage to the kernels. After the seed corn kernels are dried it is carefully shelled to avoid damage. Unlike feed corn, which is stored in grain elevators, seed corn kernels are graded into measured sizes to insure uniformity. Broken seeds, diseased seeds, light low mass kernels, chaff, foreign material, etc. are removed from seed corn. Bulk feed corn, however, includes many broken and damaged kernels, foreign material, chaff, dust, diseased kernels, insects, etc.

Uniform sizing of the corn kernels is an advantage when using the kernels in a heating pad. Kernels that are smaller than the other kernels tend to overheat and burn and thereby create a pungent burning odor. When overheated, the smaller kernels may become damaged thereby affecting their ability to retain heat.

As noted above, seed corn is cleaned and sorted to only include whole kernels that are not broken open. Because seed corn kernels are whole and unbroken, they maintain higher moisture content, and therefore produce superior moist heat for therapeutic use while retaining and emitting heat longer. The inventor has discovered that broken kernels easily become scorched and smell bad quickly, while whole kernels do not. Further, since seed corn is free of dust and chaff, it has very little smell. Also, because kernels are whole, seed corn makes a superior, long-lasting reusable product. In addition, seed corn is more resistant to mold and mildew than is feed corn. For these reasons, seed corn was found to be unexpectedly much more advantageous than feed corn despite its considerably higher price.

Relative to known forms of feed corn, the corn used in the present invention is substantially free of dust, disease, insects, rocks and foreign matter. Although seed corn is preferred, other forms of corn that have been more carefully screened and treated may have much of the preferred qualities of seed corn when used as a heating or cooling pad. Accordingly, the present invention is not necessarily limited to seed corn, but may include any form of corn that is substantially free of dust, disease, insects, rocks and foreign matter.

The therapeutic device 10 is used for the purpose of heating or freezing the seed corn filled bag 12 and applying it directly to the body for therapeutic use, for the purpose of relieving pain, reducing stress, relaxing muscles, reducing swelling, or providing overall general relaxation and enjoyment. The therapeutic device 10 provides a new, unique and effective way to relieve pain and muscle soreness, to relieve tension, and to provide an overall sense of warmth, enjoyment and comfort, achieved by heating or cooling the corn bag 10, and applying it directly to the body.

The therapeutic device 10 is used to apply heat by heating the bag 10 in a microwave oven for two to three minutes with proper rotation. The heating time may vary depending on the wattage of the microwave oven. The therapeutic device 10 should not be overheated and a light fingertip touch can be applied to the device 10 prior to full application to the skin to test the temperature of the device 10.

The device 10 is a therapeutic health and personal care item. This invention is distinguished from other products by its use of seed corn as a heat conductor and transmitter, and the encasement of the bag 12 in a cloth pillowcase 14 for the direct therapeutic application of moist penetrating heat to the body. The moist heat that the seed corn emanates distinguishes it from other comparable therapeutic products, such as electric heating pads and silicone packs. The most unique and significant improvement seed corn bags possess over existing products is penetrating moist heat, a characteristic not found in heating pads or silicone packs. Seed corn, because of the density and weight of the unit, is an excellent conductor of both heat and moisture, and the seed corn kernels have a unique ability to retain and transmit both heat and moisture for long periods of time. An improvement over existing products includes reduced purchase cost versus heating pads and silicone packs. Reduced usage cost is also a feature, when compared to electrical costs of using a heating pad.

An additional improvement over current products is that the device 10 may be heated and taken to bed without the fear of electrical fires associated with heating pads, or possible burns, also associated with bed use of heating pads. This is a significant benefit to those suffering from chronic pain or who are bedridden and must seek relief daily for long periods of time. It is also a benefit to those who may currently routinely fall asleep while using heating pads in bed, risking burns to the skin, or possible fire hazards.

The device 10 may be reused indefinitely without an appreciable loss of heat or moisture quality. The device 10 is also unique and novel in its aesthetic application: the decorative cover 14 of designer-quality cotton/flannel distinguishes it from unattractive heating pads and silicone packs.

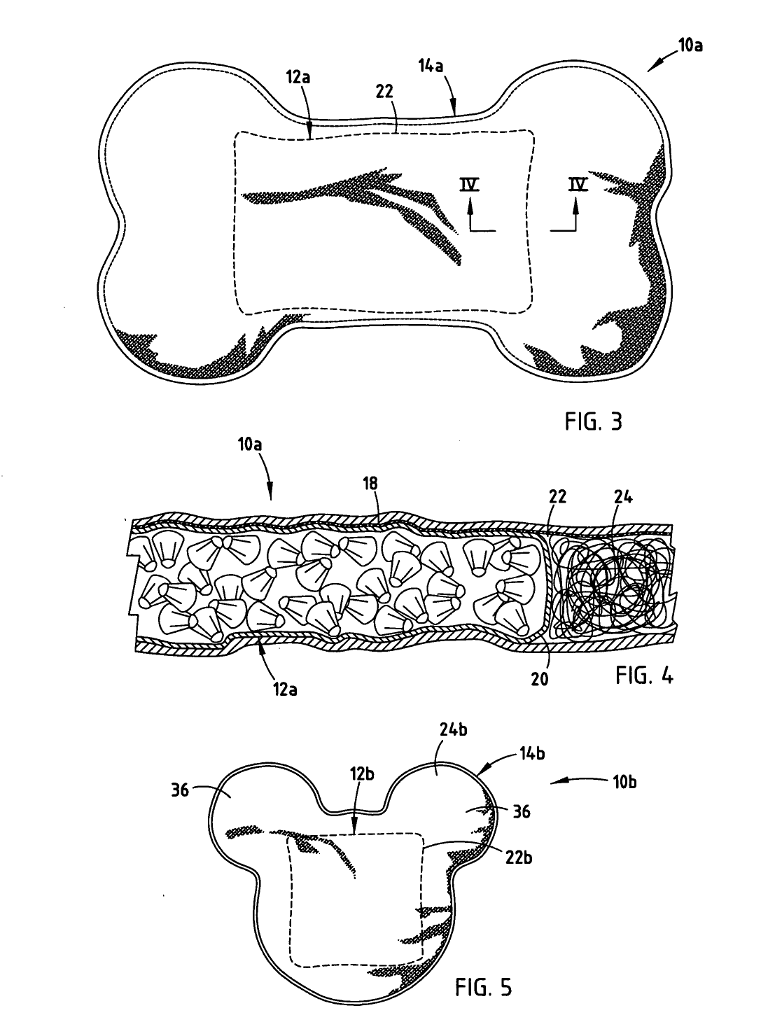

The reference numeral 10a (FIGS. 3-4) generally designates another embodiment of the present invention, having a second embodiment for the therapeutic device. Since the therapeutic device 10a is similar to the previously described therapeutic device 10, similar parts appearing in FIG. 1-2 and FIGS. 3-4, respectively, are represented by the same, corresponding reference number, except for the suffix “a” in the numerals of the latter. The therapeutic device 10a includes the enclosed member or bag 12a as the first embodiment of the therapeutic device 10, but with a differently designed cover 14a. The cover 14a may include a washable liner 18 located between the cloth 20a and the enclosed member 12a. The cover 14a of the therapeutic device 10a is dog-boned shaped and includes a rectangular center open portion 22 for accepting the enclosed member 12a and soft material 24 surrounding the rectangular center open portion 22 and defining the dog-bone shape of the cover 14a. The soft material 24 is preferably cotton or any other filler typically used in a pillow. The therapeutic device 10a can have shapes other than the dog-bone shape and is preferably used as a dog bed.

The reference numeral 10b (FIG. 5) generally designates another embodiment of the present invention, having a third embodiment for the therapeutic device. Since the therapeutic device 10b is similar to the previously described therapeutic device 10a, similar parts appearing in FIG. 1-2 and FIG. 5, respectively, are represented by the same, corresponding reference number, except for the suffix “b” in the numerals of the latter. The therapeutic device 10b includes the enclosed member or bag 12a as the second embodiment of the therapeutic device 10a, but with a differently designed cover 14b. Preferably, the cover 14b is shaped like a head 34 and includes the rectangular center open portion 22b for accepting the enclosed member 12a and soft material 24b surrounding the rectangular center open portion 22b and defining rest of the head 34, including ears 36.

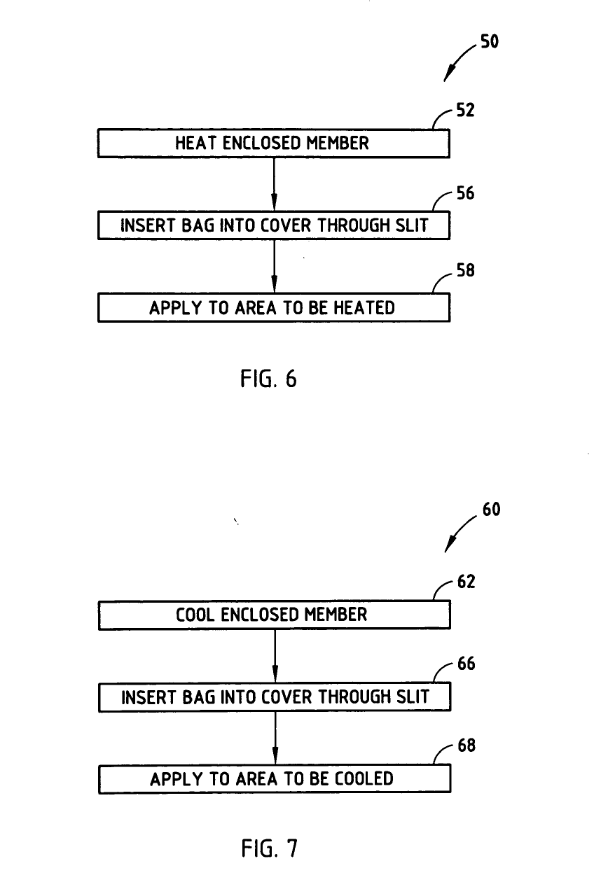

The illustrated therapeutic device 10 of the present invention relieves pain, reduces stress, relaxes muscles, reduces swelling, or provides overall general relaxation and enjoyment. Referring to FIG. 6, a method 50 of applying heat is shown. Beginning at step 52 of the method 50 of applying heat, the enclosed member 12 is heated, preferably in a microwave, to heat the plurality of seed corn therein. Thereafter, the enclosed member 12 is placed within the cover 14 by inserting the cover 14 into the slit 16 in the cover 14 at step 56. Finally, the cover 14 with the enclosed member 12 therein is applied to an area to be heated at step 58.

The illustrated therapeutic device 10 of the present invention relieves pain, reduces stress, relaxes muscles, reduces swelling, or provides overall general relaxation and enjoyment. Referring to FIG. 7, a method 60 of applying cold is shown. Beginning at step 62 of the method 60 of applying cold, the enclosed member 12 is cooled to cool the plurality of seed corn therein. Thereafter, the enclosed member 12 is placed within the cover 14 by inserting the cover 14 into the slit 16 in the cover 14 at step 66. Finally, the cover 14 with the enclosed member 12 therein is applied to an area to be cooled at step 68.

In the forgoing description, it will be readily appreciated by those skilled in the art that modifications may be made to the invention without departing from the concepts disclosed herein. Such modifications are to be considered as included in the following claims, unless these claims by their language expressly state otherwise.

What is claimed is:

1. A therapeutic device comprising:
an enclosed member having a plurality of kernels of seed corn therein; and
a cover enclosing the enclosed member,
wherein the seed corn is capable of being exothermic when heated or endothermic when cooled.

2. The therapeutic device of claim 1, wherein:
the cover has a slit for the insertion and removal of the enclosed member, and the enclosed member can be removed from the cover and the cover can be washed to clean the cover.

3. The therapeutic device of claim 1, wherein:
the cover is comprised of cloth.

4. The therapeutic device of claim 3, wherein:
the cover further includes a washable liner located between the cloth and the enclosed member.

5. The therapeutic device of claim 1, wherein:
the cover is dog-boned shaped and includes a rectangular center open portion for accepting the enclosed member and a soft material surrounding the rectangular center open portion and defining the dog-bone shape of the cover.

6. The therapeutic device of claim 3, wherein:
the cover includes a decorative design thereon.

7. The therapeutic device of claim 3, wherein:
the cover is head-shaped and includes a rectangular center open portion for accepting the enclosed member and a soft material surrounding the rectangular center open portion and defining the head.

8. A therapeutic device comprising:
a casing; and
a plurality of kernels of seed corn disposed in said casing.

9. The therapeutic device of claim 8, wherein said casing is an enclosed member.

10. The therapeutic device of claim 9, and further comprising a cover enclosing the enclosed member, the cover having a slit for the insertion and removal of the enclosed member.

11. The therapeutic device of claim 10, wherein:
the cover is comprised of cloth.

12. The therapeutic device of claim 11, wherein:
the cover further includes a washable liner located between the cloth and the enclosed member.

13. The therapeutic device of claim 10, wherein:
the cover is dog-boned shaped and includes a rectangular center open portion for accepting the enclosed member and a soft material surrounding the rectangular center open portion and defining the dog-bone shape of the cover.

14. The therapeutic device of claim 11, wherein:
the cover includes a decorative design thereon.

15. The therapeutic device of claim 11, wherein:
the cover is head-shaped and includes a rectangular center open portion for accepting the enclosed member and a soft material surrounding the rectangular center open portion and defining the head.

16. A therapeutic device comprising:
a casing; and
corn matter disposed in said casing,
wherein said corn matter includes a plurality of kernels of corn and is substantially free of dust, disease, insects, rocks and foreign matter.

17. The therapeutic device of claim 16, and further comprising a cover, wherein:
said casing is an enclosed member,
said cover encloses the enclosed member, and
said cover has a slit for the insertion and removal of the enclosed member.

18. The therapeutic device of claim 1, wherein:
the cover is comprised of cloth.

19. The therapeutic device of claim 18, wherein:
the cover further includes a washable liner located between the cloth and the enclosed member.

20. The therapeutic device of claim 18, wherein:
the cover includes a decorative design thereon.
